(19) United States
(12) Patent Application Publication
(10) Pub. No.: US 2024/0307089 A1
(43) Pub. Date: Sep. 19, 2024

(54) ULTRASOUND IMAGING MULTI-ARRAY PROBE APPARATUS AND SYSTEM

(71) Applicant: Rivanna Medical, Inc., Charlottesville, VA (US)

(72) Inventors: Frank William Mauldin, Troy, VA (US); Adam Dixon, Charlottesville, VA (US); Zachary Leonard, Charlottesville, VA (US); Paul Sheeran, Waxhaw, NC (US); Kathryn Ozgun, Charlottesville, VA (US); Qikun Wang, Charlottesville, VA (US); Gerd Schmieta, Boston, MA (US); James Pelletier, Westhampton, MA (US)

(21) Appl. No.: 18/608,412

(22) Filed: Mar. 18, 2024

Related U.S. Application Data
(60) Provisional application No. 63/452,920, filed on Mar. 17, 2023.

(51) Int. Cl. A61B 17/34; A61B 90/00

(52) U.S. Cl. CPC A61B 17/3403; A61B 90/37; A61B 2017/3413; A61B 2090/378

(57) ABSTRACT

An ultrasound-based scanning apparatus using multiple transducer arrays and a physical gap for guidance and insertion of a medical instrument, such as a needle, and methods to detect and visualize the inserted medical instrument within a target region of the patient anatomy.

### CROSS-REFERENCE TO RELATED APPLICATION

The present application relies on the disclosures of and claims priority to and the benefit of the filing date of the following U.S. Patent Application 63/452,920, filed Mar. 17, 2023. The disclosures of that application are hereby incorporated by reference herein in their entireties.

### STATEMENT REGARDING FEDERALLY SPONSORED RESEARCH

This invention was made with government support under Grant No. R44NS120798 awarded by the National Institutes of Health (NIH) National Institute of Neurological Disorders and Stroke (NINDS). The government has certain rights in the invention.

### TECHNICAL FIELD

The present invention is related to an ultrasound-based scanning device and more specifically to an apparatus and method for using ultrasound-based scanning to assist needle injection procedures.

### BACKGROUND OF THE INVENTION

Interventional procedures in medicine are numerous and comprise many different procedures including, for example, lumbar punctures, bone marrow biopsies, acute pain analgesia, and chronic pain therapy injections. The techniques available for interventional guidance range from a palpation-based approach, where no image guidance is utilized, to guidance with ultrasound imaging, computed tomography, or fluoroscopy. The palpation approach is low-cost and accessible at the bedside but suffers from low procedure success rates and higher rates of complications. Conventional ultrasound can improve success rates, and is utilized in some instances, but suffers from limitations including an extended learning curve and workflow barriers resulting from the need to simultaneously manipulate an ultrasound probe and insert a medical instrument, such as a needle, that typically requires the use of two hands. X-ray-based approaches, such as computed tomography or fluoroscopy, exhibit high success rates but expose the patient to ionizing radiation and increase procedure cost and are generally inaccessible at the bedside or incompatible with workflow constraints in fields such as emergency medicine.

To overcome the limitations of current state of the art approaches to interventional procedure guidance, and specifically for medical needle guidance procedures, the present invention describes a unique ultrasound-based apparatus for both two-dimensional and three-dimensional scanning, with a physical separation between the arrays that may act as or comprise a guide for a medical instrument, such as a needle. Herein, the apparatus enables needle advancement in-plane with a real-time and/or simultaneous ultrasound image acquisition from multiple ultrasound transducer arrays. The invention retains the benefits of medical ultrasound while addressing common workflow barriers that reduce utilization.

Related art describes apparatus to facilitate interventional procedures involving medical instruments.

PCT Application No. PCT/JP2014/050941, hereby incorporated by reference herein, describes a system comprising an ultrasound probe and puncture needle, the ultrasound probe having a wedge-shaped configuration so that a single ultrasound transducer array housed inside the probe is tilted at an angle relative to the body, providing ultrasound probe configured to be angled relative to patient anatomy. As it relates to the current invention described herein, the system of PCT/JP2014/050941 does not support two or more arrays and does not permit an in-plane midline needle trajectory. Moreover, the current invention includes components configured to reduce the presence of acoustic reverberations within the probe housing resulting from an angled transducer array.

PCT Application No. PCT/CA2009/001700, hereby incorporated by reference herein, describes an ultrasound imaging and medical instrument guiding apparatus comprising two ultrasound probes configured on a mount to acquire distinct 3-dimensional images of overlapping volumes and a positionable medical instrument guide that allows propagation of the medical instrument into the overlapped region of the imaging volumes from the two ultrasound probes. However, the configuration of PCT/CA2009/001700 differs from the current invention described herein, and in embodiments of the current invention described herein, the apparatus comprises two or more angled ultrasound transducer arrays, each array having, in embodiments, an acoustic standoff that enables angling of the ultrasound transducer array, which again, among other reasons, is unlike PCT/CA2009/001700.

U.S. application Ser. No. 06/396,784, hereby incorporated by reference herein, describes an ultrasonic probe for use in needle insertion procedures, the ultrasonic probe including a support having an array of ultrasonic transducer elements lying flatwise on the front end and a groove in the support for guiding the needle. In the ultrasonic probe of U.S. Ser. No. 06/396,784, the groove forms an opening at the front end of the support, and one or more transducer elements are located adjacent the opening of the groove and between the other transducer elements, thus leaving no blank space on the front end of the support. In differentiation with the ultrasonic probe of U.S. Ser. No. 06/396,784, among other reasons, the current invention comprises two or more angled ultrasound transducer arrays, each array having, in embodiments, an acoustic standoff that enables angling of the ultrasound transducer array. Furthermore, the current invention described herein comprises ultrasound transducer arrays that produce overlapping 2D images. Finally, the current invention comprises a configuration that enables a needle angulation of up to 20 degrees away from the central axis of the needle guide, by way of example, and is overall an improvement over U.S. Ser. No. 06/396,784, as well as the other related art.

JP7153980A, hereby incorporated by reference herein, describes an ultrasonic probe comprising two flat ultrasound transducer arrays having a groove between the two flat ultrasound transducer arrays, and further requires a cannula (needle) placed parallel to the primary axis of the groove. In differentiation with the ultrasonic probe of JP7153980A, the current invention described herein comprises two or more angled ultrasound transducer arrays, each array having, in embodiments, an acoustic standoff that enables angling of the ultrasound transducer array. Furthermore, the current invention comprises ultrasound transducer arrays that produce overlapping 2D images. Finally, the current invention comprises a configuration that enables a needle angulation of up to 20 degrees away from the central axis of the needle guide, by way of example, and is overall an improvement over JP7153980A, as well as the other related art

U.S. application Ser. No. 06/511,285 hereby incorporated by reference herein, describes an ultrasonic transducer probe comprising a flat ultrasound transducer array with a gap that can receive a removeable wedge-shaped cannula (needle) adapter. In differentiation with the ultrasonic probe of U.S. Ser. No. 06/511,285, the current invention described herein comprises two or more angled ultrasound transducer arrays, each array having, in embodiments, an acoustic standoff that enables angling of the ultrasound transducer array. Furthermore, the current invention comprises ultrasound transducer arrays that produce overlapping 2D images. Finally, the current invention comprises a configuration that enables a needle angulation of up to 20 degrees away from the central axis of the needle guide, by way of example, and is overall an improvement over U.S. application Ser. No. 06/511,285, as well as the other related art.

U.S. application Ser. No. 06/014,076 hereby incorporated by reference herein, describes an ultrasonic transducer probe comprising ultrasonic transducer elements arranged proximate to a surface that is positioned on the body surface of a subject, further comprising a shaped cavity that provides a guide block for a cannula (needle) while also allowing for removal of the ultrasonic transducer probe from the inserted canula, the guide block being sterilizable after removable. In differentiation with the ultrasonic probe of U.S. Ser. No. 06/014,076, the current invention comprises two or more angled ultrasound transducer arrays, each array having, in embodiments, an acoustic standoff that enables angling of the ultrasound transducer array. Furthermore, the current invention comprises ultrasound transducer arrays that produce overlapping 2D images. Finally, the current invention comprises a configuration that enables a needle angulation of up to 20 degrees away from the central axis of the needle guide, by way of example, and is overall an improvement over U.S. application Ser. No. 06/014,076, as well as the other related art.

GB0307311A, hereby incorporated by reference herein, describes an ultrasound probe comprising a housing and guide for needle insertion, the guide comprising a channel located between ultrasound transducers in the housing. In differentiation with the ultrasonic probe of GB0307311A, the current invention described herein comprises two or more angled ultrasound transducer arrays, each array having, in embodiments, an acoustic standoff that enables angling of the ultrasound transducer array. Additionally, the current invention describes a configuration that enables a needle angulation of up to 20 degrees away from the central axis of the needle guide. Finally, the current invention specifies components configured to reduce the presence of acoustic reverberations within the probe housing resulting from an angled transducer array, by way of example, and is overall an improvement over GB0307311A, as well as the other related art.

PCT Application No. PCT/US2018/026413, hereby incorporated by reference herein, describes a system comprising an ultrasound probe, the ultrasound probe comprising two ultrasound transducers arranged at an angle that transmit sound waves to create an overlapping imaging region, and a detachable needle guide disposed between the two transducers that extends toward a target location in the overlapping imaging region. In differentiation with the ultrasonic probe of PCT/US2018/026413, the current invention described herein comprises two or more angled ultrasound transducer arrays, each array having, in embodiments, an acoustic standoff that enables angling of the ultrasound transducer array. Additionally, the current invention describes a configuration that enables a needle guide integral to the probe housing that allows needle angulation of up to 20 degrees away from the central axis of the needle guide. Finally, the current invention specifies components configured to reduce the presence of acoustic reverberations within the probe housing resulting from an angled transducer array, by way of example, and is overall an improvement over PCT/US2018/026413, as well as the other related art.

### SUMMARY OF THE INVENTION

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

In embodiments, the present invention overcomes limitations of existing interventional procedure guidance systems by providing a form factor with multiple arrays that allows a central, through-probe trajectory to insert a medical instrument, such as a needle. The unique form factor enables real-time ultrasound acquisition with a midline approach, which is often preferred for neuraxial needle guidance procedures such as lumbar punctures or epidurals.

In embodiments, the dual-array probe may act as or comprise a needle guide which guides the medical instrument trajectory through the probe or adjacent to the probe and enables midline, paramedian, or oblique needle approaches.

In embodiments, the present invention includes one or more mechanical apparatus for use with the multi-array probe that provides a detachable component to retain the medical instrument or needle with latching function to grip the needle and enable midline, paramedian, or oblique needle approaches.

In embodiments, the present invention enables multi-angle, multi-array compounding and filtering which can be used to improve the ultrasound imaging visualization of bony anatomies, vascular anatomies, and inserted medical instruments, such as needles.

In embodiments, the present invention includes sensors in the probe to enable position-registered ultrasound data acquisition and volumetric reconstruction.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIG. 1 is a schematic illustration of an exemplary ultrasound system that incorporates a multi-array ultrasound probe, according to an embodiment of the invention described herein.

FIG. 2 is a schematic illustration of an exemplary multi-array ultrasound probe, according to an embodiment of the invention described herein.

FIGS. 3A-3D are schematic illustrations of an exemplary multi-array ultrasound probe and of an apparatus that affixes to the multi-array ultrasound probe to provide needle retention functionality, according to an embodiment of the invention described herein.

FIG. 4A-4C depict an exemplary ultrasound scan geometry enabled by the multi-array ultrasound probe and exemplary depictions of multi-angle acquisition and image compounding processes to enable needle and bone visualization, according to an embodiment of the invention described herein.

FIGS. 5A-5C depict an exemplary approach for simultaneously acquiring ultrasound image data and position tracking data to support volumetric reconstruction, according to an embodiment of the invention described herein.

FIG. 6 depicts a flow diagram that describes an exemplary method of multi-angle compounding for needle detection and enhancement, according to an embodiment of the invention described herein.

FIG. 7 depicts a flow diagram that describes an exemplary method of differentiating between needles and bones during multi-angle compounding and image rendering, according to an embodiment of the invention described herein.

FIGS. 8A-8B depict a flow diagram of a process by which the present apparatus with an exemplary dual-array ultrasound probe with or without position tracking may be used by a clinician to assist in an interventional procedure, according to an embodiment of the invention described herein.

FIGS. 9A-9B depict a schematic illustration and an exploded component view of an exemplary multi-array ultrasound probe separated from the patient anatomy by intervening acoustically transmissive layers, according to an embodiment of the invention described herein.

FIG. 10 depicts a schematic illustration of an exemplary multi-array ultrasound probe separated from the patient anatomy by intervening acoustically transmissive layers, with grip, button placement, and housing design configured to provide unobstructed view(s) of the medical instrument, according to an embodiment of the invention described herein.

FIG. 11 depicts a schematic illustration of an exemplary multi-array ultrasound probe and acoustic field of view relative to a needle.

### DETAILED DESCRIPTION OF VARIOUS EMBODIMENTS OF THE INVENTION

Ultrasound imaging transducer assemblies, otherwise known as ultrasound transducer arrays, are used in a variety of medical or clinical applications to enable medical imaging functions. In this non-limiting example, an ultrasound transducer is disposed within a transducer array to deliver a pulse, tone, sequence, or programmed energy signal into a target location to be imaged. A specific example is one or more ultrasound transducer elements that deliver an ultrasound signal into a patient's body and detect a return signal so as to form a computer-generated image of the target region. Different ultrasound imaging modes can be utilized, depending on a given application and design as known to those skilled in the art. The present disclosure can be used in medical ultrasound applications but is not limited to this application. Those skilled in the art will appreciate that a variety of types of transducers, signal transmitters and/or receivers, and other arrays can also benefit from the present invention, which are comprehended hereby. The preferred embodiments herein describe needle guidance. Those skilled in the art will appreciate that the present invention may be used to guide a variety of medical instruments including, but not limited to, a catheter, trocar, ablation instrument, cutting instrument, or therapy applicator. The present invention can be utilized, in a preferred embodiment, with systems and methods previously disclosed by Mauldin et al. (PCT/US2019/012622), which is incorporated by reference herein, for automated three-dimensional detection, guidance, and visualization of ultrasound-based therapy guidance procedures.

In the embodiment of the present invention for medical applications of needle guidance described herein, a probe containing two or more ultrasound transducer arrays, otherwise termed a multi-array probe, is described. An objective of the multi-array ultrasound probe is to provide access for a medical instrument, such as a needle, to pass through the center of the probe silhouette on the patient body, at an angle of incidence that is approximately orthogonal to the patient's skin. In the embodiment of the present invention, the medical instrument trajectory may be described as ‘in-plane’, wherein the point of insertion transects the long axis of the probe housing, ‘midline’, wherein the insertion trajectory aligns with the axis of symmetry of the spine vertebral body, or ‘paramedian’, wherein the insertion trajectory is performed at an angle relative to the axis of symmetry of the spine vertebral body. In embodiments, additional objectives include algorithmic contrast enhancement of the needle within the ultrasound image, differentiation between the needle and bright anatomical structures in the ultrasound image (i.e., bone), and three-dimensional ultrasound scanning to facilitate assessment of large sections of patient anatomy. In embodiments, images acquired from each of the ultrasound transducer arrays in the multi-array probe may be compounded, such as by simple averaging, weighted averaging, or based on measurements of similarity to form composite images with a wider field of view and containing an overlapping image region.

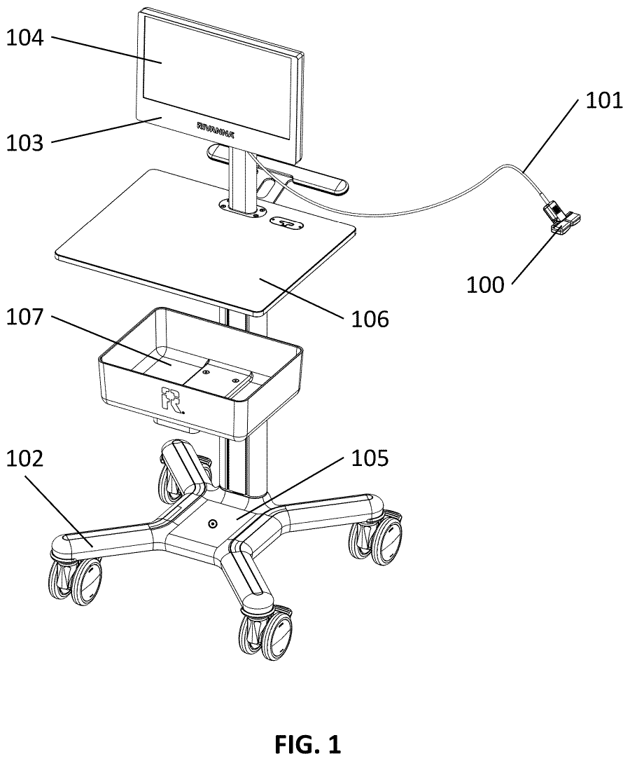

In one exemplary embodiment depicted in FIG. 1, the multi-array ultrasound probe 100 can be connected by an electrical signal cable 101 to a mobile cart 102 to allow the imaging device to be moved to the bedside and positioned at the required orientation for acquiring images of the patient's anatomy. The cart 102 includes an enclosure 103 which may contain a computer processor and monitor 104, battery 105, and other associated electronics familiar to those skilled in the art which are needed to power and communicate with the imaging device 100. The cart 102 can be outfitted with additional input/output devices such as a keyboard, mouse, or monitor 104, which may also be a touchscreen display. The cart can be outfitted with a worksurface 106 and compartment 107 in order to place or store materials commonly used during interventional procedures, such as consumables, needles, ultrasound gel, and disinfectant wipes. The monitor 104 may be positionally adjustable about the cart in order to orient the imaging device 100 and monitor 104 in various relative positions, heights, and orientations for the needle guidance procedure. In a preferred embodiment, the enclosure 103 may simultaneously contain the monitor 104, the computer processor, and the ultrasound front-end electronics. A computer processor within the enclosure 103 may be used to perform ultrasound signal and image processing steps required to form an ultrasound image reconstruction that can be displayed on the monitor 104. Such processing steps are known to those skilled in the art of medical ultrasound and may include: beamforming, bandpass filtering, scan conversion, spatial compounding, Doppler imaging, and image rendering. Two- and three-dimensional images may be rendered using various techniques, including simultaneous display, as described in Mauldin et al., U.S. Pat. No. 11,504,095, which is hereby incorporated herein by reference. In a preferred embodiment, the computer processor in the enclosure 103 receives signals from the ultrasound probe 100 indicating the position of the probe on the patient, which are used for interpreting spatial position of the real-time image data acquired. In a preferred embodiment, the registration of spatial position of the imaging data is used to reconstruct and render three-dimensional ultrasound images on the monitor 104 that are functionally equivalent to fluoroscopic imaging of skeletal anatomy.

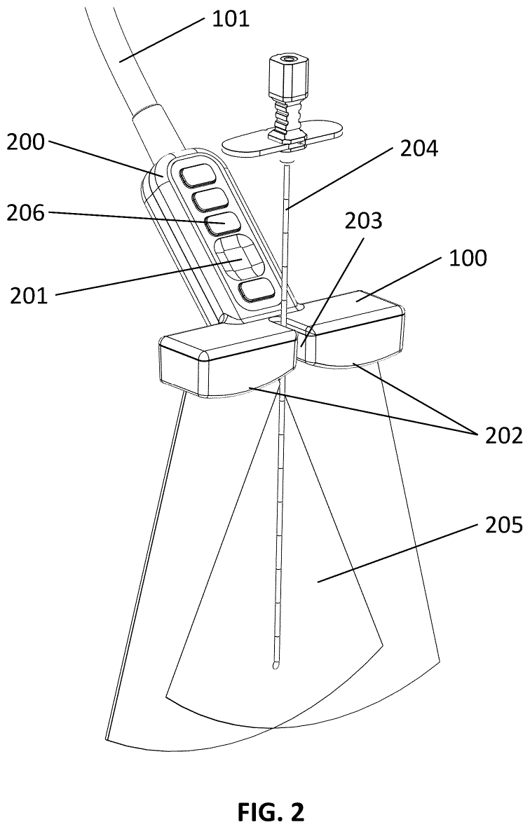

In one exemplary embodiment, the multi-array ultrasound probe 100 is depicted in FIG. 2. The multi-array probe 100 has a housing component that provides a handle 200 with grip features 201 for holding the probe. The multi-array probe 100 incorporates two ultrasound arrays 202 that interface to ultrasound data acquisition electronics via integrated circuits and an electrical signal cable 101 to acquire ultrasound image datasets. The ultrasound arrays 202 of the multi-array probe are separated by a medical instrument guide 203 comprising a gap integral to the probe housing that provides access for a central needle trajectory 204 that passes between the two ultrasound arrays 202. In this imaging configuration, the fields of view (FOV) 205 of the two ultrasound arrays 202 overlap along the needle trajectory 204, providing for independent views of the needle from two different vantage points. In some embodiments, the medical instrument guide 203 provides alignment to keep the central needle trajectory 204 in plane with the fields of view (FOV) 205 of the two ultrasound arrays while also minimizing forces imparted to the instrument, which may be advantageous for clinical procedures where the clinician relies on the tactile feedback of the instrument passing through the anatomy, such as for detecting loss of resistance. In some embodiments, the medical instrument guide 203 allows a needle angulation of up to 20 degrees relative to the central axis of the medical instrument guide 203. The dual-array probe 100 incorporates buttons 206 to provide control over imaging functionality. The handle 200 includes a printed circuit board which provides a microprocessor that interfaces to buttons 206, provides a motion sensor integrated circuit, and enables digital serial communication through the probe cable 101 to the host processor of the ultrasound imaging system.

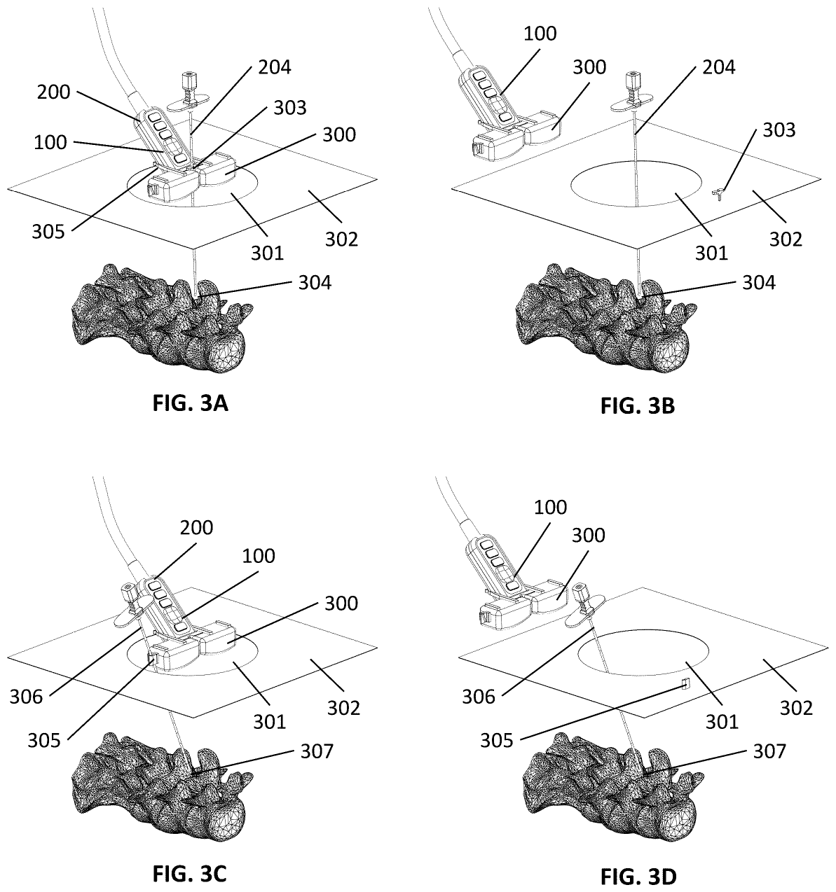

In one exemplary embodiment depicted in FIG. 3A, the multi-array probe 100 may conform with a sterile workflow in which the dual-array probe 100 is covered by a sterile sheath. The multi-array probe 100 is applied over a sterilized portion of the patient anatomy 301 to support sterile imaging interrogation, and in this embodiment a midline needle insertion is depicted. A sterile needle retention component 300 is temporarily affixed to the multi-array probe 100 and guides the central needle trajectory 204 within the apparatus and facilitates in-plane, midline needle insertion to the target anatomy 304, which in this example is the epidural space of the spine. The non-sterile patient anatomy may be covered by a sterile drape 302 (e.g., procedural drape) while the sterilized portion of the patient anatomy 301 is uncovered. The needle retention component 300 incorporates attachment features 305 that grip to the dual-array probe 100 housing and handle 200 to constrain motion of the sterile sheath.

In a non-limiting embodiment depicted in FIG. 3B, the sterile needle retention component 300 incorporates a quick-release feature 303 that permits release of the midline needle 204 and allows the probe to be moved away from the location of needle insertion while the needle 204 remains positioned at the target anatomy 304. The quick-release feature 303 may be a detachable part or integrated into the sterile needle retention component 300 and may provide a needle release mechanism such as sliding, rotation, unsnapping, or other methods familiar to those skilled in the art.

In a non-limiting embodiment depicted in FIG. 3C, the multi-array probe 100 may conform with a sterile workflow for a paramedian needle insertion in which the dual-array probe 100 is covered by a sterile sheath and a sterile needle retention component 300 is temporarily affixed to the multi-array probe 100. The multi-array probe 100 is applied over a sterilized portion of the patient anatomy 301 to support sterile imaging interrogation and paramedian needle insertion. The non-sterile patient anatomy may be covered by a sterile drape 302 (e.g., procedural drape) while the sterilized portion of the patient anatomy 301 is uncovered. The needle retention component 300 incorporates a lateral quick-release feature 305 on the short axis of the probe that guides the paramedian needle trajectory 306 lateral to the apparatus and facilitates in-plane, paramedian needle insertion to the target anatomy 307, which in this example is the epidural space of the spine.

In a non-limiting embodiment depicted in FIG. 3D, the sterile needle retention component 300 incorporates a lateral quick-release feature 305 that permits release of the paramedian needle 306 and allows the probe to be moved away from the location of needle insertion while the needle 306 remains positioned at the target anatomy 307. The lateral quick-release feature 305 may be a detachable part or integrated into the sterile needle retention component 300 and may provide a needle release mechanism such as sliding, rotation, unsnapping, or other methods familiar to those skilled in the art.

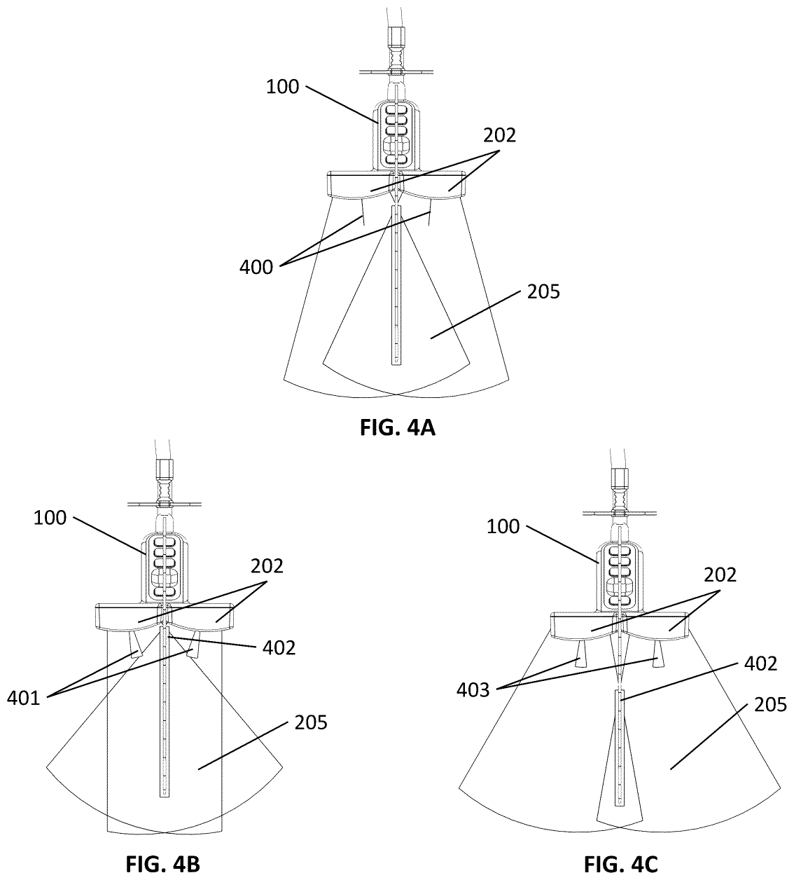

FIGS. 4A-4C depict schematic illustrations of the scanning field of view 205 of the multi-array probe 100 when multi-angle beam steering is applied. In FIG. 4A, the fields of view 205 of the ultrasound arrays 202 are forward looking with a neutral (0-degree) steering angle 400, whereas in FIG. 4B the fields of view 205 are angled towards the center axis of the probe with an inward (+θ) steering angle 401 to increase field of view 205 overlap in the region of needle placement 402. In FIG. 4C, the fields of view 205 are angled away from the center axis of the probe with an outward (−θ) steering angle 403 to minimize field of view 205 overlap in the region of needle placement 402. In this exemplary embodiment, the beam steering of each of the individual arrays may be controlled independently. The main processor of the ultrasound imaging system may render two-dimensional images of each individual acquisition or may geometrically compound the acquisitions to combine information in the overlapping regions of the field of view 205, an approach known to those skilled in the art as spatial compounding.

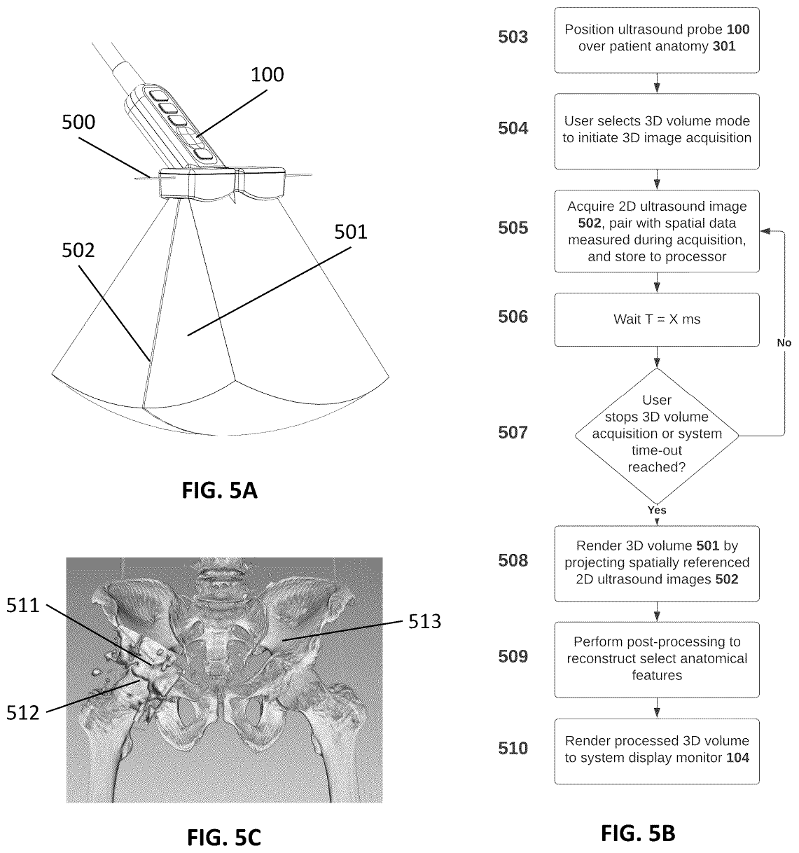

In a non-limiting embodiment depicted in FIG. 5A, the clinician may adjust the position of the multi-array ultrasound probe 100 during scanning to collect a series of motion-tracked ultrasound images, with motion information recorded for each two-dimensional ultrasound image by the motion sensor integrated circuit and digitized and communicated to the host processor of the ultrasound system by the microprocessor in the multi-array probe 100. In this exemplary embodiment, the multi-array probe 100 is swept about an axis 500 to generate a three-dimensional dataset 501 using the recorded motion data from each two-dimensional ultrasound image 502. The multi-array probe 100 may be translated or rotated about alternative axes in order to acquire a three-dimensional dataset of a wide range of shapes which may be advantageous for specific image-guided procedures, such as a translating the multi-array probe 100 on a linear path along the spine for neuraxial needle guidance procedures.

The host processor of the ultrasound system can render geometrically accurate volumetric renderings using the motion tracking information to project the two-dimensional images 502 into a three-dimensional rendering space by following the exemplary flow diagram that is depicted in FIG. 5B. First, at block 503 the user positions the multi-array ultrasound probe 100 on the patient over the patient anatomy 301. Next, at block 504 the user selects the 3D imaging mode using one of the buttons 206 of the probe 100, the user interface on the monitor 104, or other user input method to initiate the three-dimensional image acquisition process. At block 505, a two-dimensional ultrasound image 502 is acquired by the electronics, paired with the spatial positioning of the probe 100 during the image acquisition as determined by the motion sensor integrated circuit, and stored to system memory on a processor. The system optionally waits an amount of time (such as a short amount of time) at block 506, then evaluates if the user has stopped the three-dimensional acquisition or the system has reached a pre-determined time limit at block 507. If the three-dimensional image acquisition process has not been halted, another two-dimensional ultrasound image 502 is acquired at block 505. If the three-dimensional image acquisition process has been halted, at block 508 the system projects the spatially referenced two-dimensional ultrasound images 502 into a geometrically accurate rendering of the three-dimensional volume 501. The rendering process may include the use of smoothing, filtering, or other methods familiar to those skilled in the art. Next, at block 509 the system performs post-processing on the two-dimensional ultrasound images 502 or three-dimensional dataset 501 to enable specific reconstruction of select anatomical features, such as blood flow (via Doppler imaging to isolate regions with blood flow), bone-only renderings (as described in Mauldin et al., U.S. Pat. No. 10,548,564, incorporated herein by reference), and similar approaches. Finally, at block 510 the system renders the processed three-dimensional dataset and displays it on the monitor 104. The three-dimensional image acquisition process can be initiated before needle insertion (scout scan) as described in FIG. 5B, during the needle insertion to assist with needle guidance, or after the needle has reached the target anatomy to verify the needle location meets the clinician's expectations (e.g., needle has been placed at the correct spinal level for a steroid injection). The three-dimensional rendering and display to the monitor 104 may alternatively be performed partially or fully contemporaneously with the three-dimensional image acquisition process such that the three-dimensional rendering is effectively displayed and updated in real-time.

In a preferred embodiment, a bone-only volumetric rendering 511 of the target anatomy 512 is depicted in FIG. 5C. In this non-limiting example, the target anatomy 512 for a peripheral nerve block is the valley in the fascial plane between the psoas muscle and the superior pubic ramus. In this non-limiting example, the bone surfaces have been extracted from the two-dimensional ultrasound images 502 via application of a bone segmentation filter, and only the bone surfaces have been projected into the volumetric rendering space. In this example, the volumetric rendering 511 of the femoral head and iliac bone has been overlaid on a pseudo-fluoroscopic volumetric rendering 513.

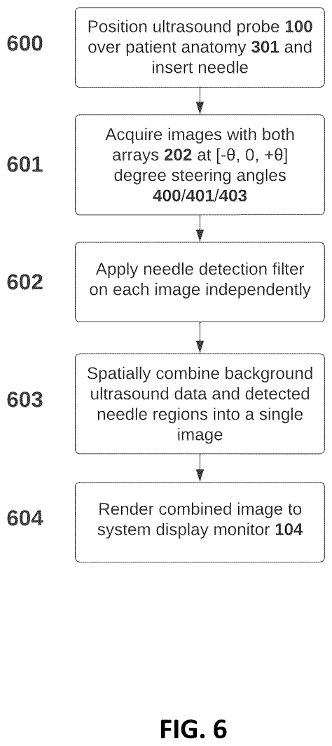

An exemplary flow diagram that describes an approach for multi-array, multi-angle needle filtering is depicted in FIG. 6. At block 600, the user positions the multi-array ultrasound probe 100 on the patient over the patient anatomy 301 and inserts the needle. Next, at block 601 the system acquires images using the two ultrasound arrays 202 at one or more beam steering angles: neutral 400, inward 401, or outward 403. At block 602, the system applies a needle detection filter independently on each image. At block 603, the system applies spatial compounding of the background ultrasound data and the regions containing the needle to produce a single image. Finally, at block 604 the system renders the combined image and displays it on the monitor 104.

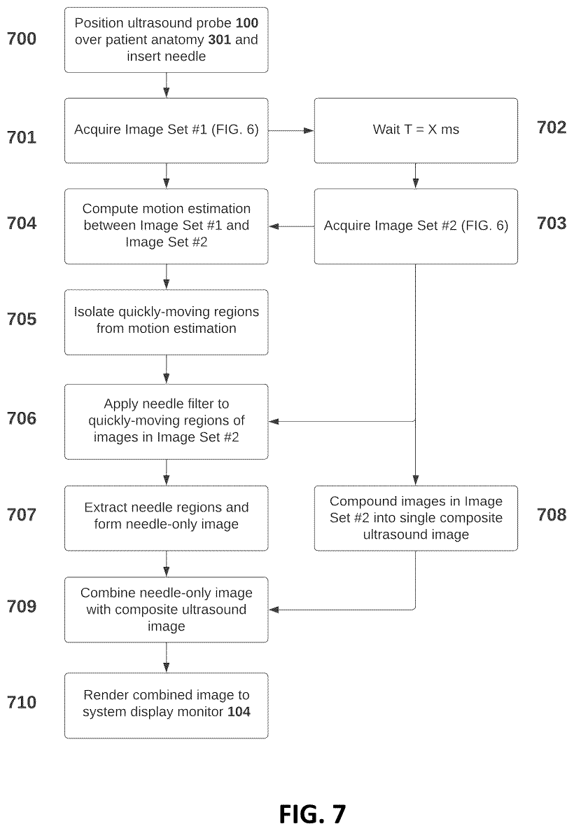

An exemplary flow diagram that describes an approach for multi-array, multi-angle needle and bone filtering is depicted in FIG. 7. This method provides for signal separation to differentiate between bright needle features and bright bone surface features in ultrasound imaging data. First, at block 700 the user positions the multi-array ultrasound probe 100 on the patient over the patient anatomy 301 and inserts the needle. At block 701, the user acquires the first image set using the methods described in FIG. 6, the system waits a time (such as 200 ms or less) at block 702, then acquires a second image set using the methods described in FIG. 6 at block 703. Next, at block 704 the system computes an estimate of motion in the ultrasound images between Image Set #1 and Image Set #2. At block 705 the system isolates regions in the ultrasound images that are quickly-moving based on the motion estimation, then at block 706 it applies a needle filter to the quickly-moving regions of the ultrasound images in Image Set #2, and then at block 707 the system extracts the needle regions from the quickly-moving regions and forms the needle-only image. In parallel, at block 708 the system performs spatial compounding on the ultrasound images in Image Set #2 and produces a single composite ultrasound image. Next, at block 709 the system combines the needle-only image of block 707 with the composite ultrasound image of block 708. Finally, at block 710 the system renders the combined image and displays it on the monitor 104.

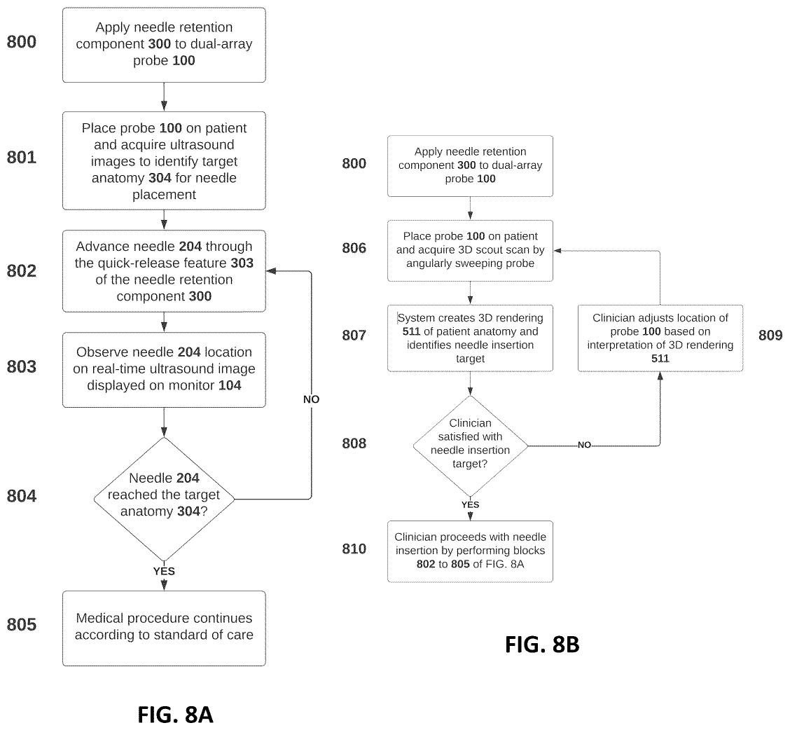

An exemplary flow diagram that describes a process by which a clinician would use the invention to perform a needle placement procedure is depicted in FIG. 8A. First, at block 800 the user applies the sterile needle retention component 300 to the multi-array ultrasound probe 100. Next, at block 801 the user places the multi-array probe 100 on the patient and acquires ultrasound images to identify the target patient anatomy 304 for needle placement. Once the user identifies the target patient anatomy 304, at block 802 the user advances the needle 204 through the medical instrument guide 203. The quick-release feature 303 of the needle retention component 300 ensures the needle remains on the intended trajectory and in-plane of the ultrasound arrays. At block 803, the user observes the location of the needle 204 using the real-time ultrasound image display on monitor 104. At block 804, the user decides if the needle 204 has reached the target patient anatomy 304. If the needle 204 has not reached the target patient anatomy 304, the user returns to block 802 to continue adjusting the position of needle 204. Finally, if the needle 204 has successfully reached the target patient anatomy 304, at block 805 the clinician continues the medical procedure according to the standard of care.

An exemplary flow diagram that describes a process by which a clinician would use the invention to perform a needle placement procedure using a 3D scout scanning approach is depicted in FIG. 8B. First, at block 800 the user applies the sterile needle retention component 300 to the multi-array ultrasound probe 100. Next, at block 806 the user places the multi-array probe 100 on the patient and sweeps the probe 100 about an angle to acquire a scout scan of a three-dimensional dataset of the patient anatomy. At block 807, as described in FIG. 5B, the system creates a volumetric rendering 511 of the patient anatomy, identifies the target location for the needle insertion, and displays this spatial information on the monitor 104. The identification of the target location for the needle insertion may alternatively be performed by the clinician using the volumetric rendering 511. At block 808 the clinician evaluates if the current needle insertion target meets their expectations based on the needs of the medical procedure. If the current needle insertion target is not correct, at block 809 the clinician adjusts the location of the probe 100 on the patient based on their interpretation of the current target and intended locations for needle insertion on the volumetric rendering 511 and then returns to block 806. Finally, if the current needle insertion target meets the user's expectations, at block 810 the clinician proceeds with the needle insertion process as described in blocks 802 to 805 of FIG. 8A.

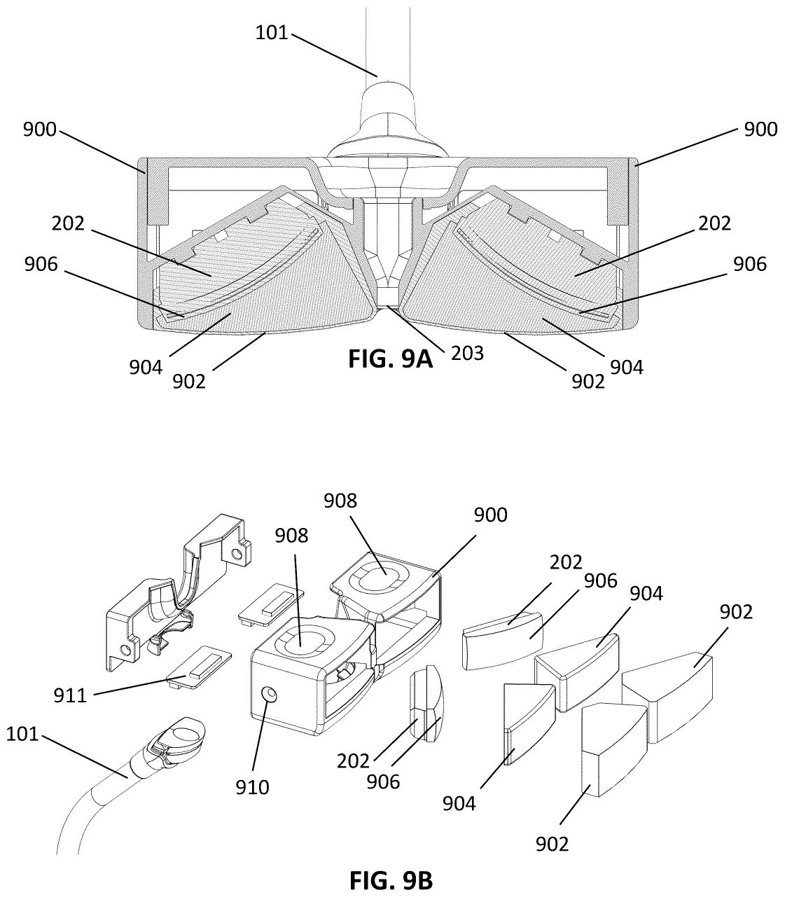

In one exemplary embodiment, the multi-array ultrasound probe is depicted in FIGS. 9A-9B. The multi-array probe has a housing component 900 containing two ultrasound arrays 202 that interface to ultrasound data acquisition electronics via integrated circuits and an electrical signal cable 101 to acquire ultrasound image datasets. The ultrasound arrays 202 of the multi-array probe are separated by a medical instrument guide 203 comprising a gap integral to the probe housing 900 that provides access for a central needle trajectory that passes between the two ultrasound arrays 202. The multi-array ultrasound probe is configured such that the two ultrasound arrays 202 do not make direct contact with the patient anatomy and are rotated within the probe housing 900 in order to provide a lower angle of incidence relative to the medical instrument guide 203 compared to the embodiment of FIG. 2. The two ultrasound transducer arrays 202 acoustically couple to the patient anatomy through three intervening acoustically transmissive layers 902, 904, and 906, although more or fewer intervening acoustically transmissive layers may be preferred. In this embodiment, the outermost acoustically transmissive layer 902 makes contact with the patient anatomy or probe sheath, and is comprised of a rigid, semi-rigid, or substantially rigid material, such as a plastic or elastomer. In this embodiment, the central acoustically transmissive layer 904 provides an acoustic ‘filler’ between the outermost transmissive layer 902 and the innermost transmissive layer 906, and may be comprised of a rigid, semi-rigid, or substantially rigid material, such as a plastic or elastomer, a deformable, semi-deformable, or substantially deformable material, such as an elastomer or a gel, or a fluid. In this embodiment, the innermost acoustically transmissive 906 layer provides a coating to protect the ultrasound transducer array, and may be comprised of a rigid, semi-rigid, or substantially rigid material, such as a plastic or elastomer, a deformable, semi-deformable, or substantially deformable material, such as an elastomer. The acoustically transmissive layers 902, 904, and 906 may be composed of materials that have low acoustic attenuation and acoustic impedance matching to the ultrasound transducer array and/or patient in order to maximize acoustic energy transmission and minimize acoustic reverberation. The material composition and fabrication methods of the acoustically transmissive layers 902, 904, and 906 may provide additional benefits over the embodiment of FIG. 2, such as greater conformability to the patient anatomy, the enablement of sterilization or high-level disinfection of the ultrasound probe, and greater durability. FIG. 9B depicts an exploded component-level view of the multi-array ultrasound probe of this embodiment. The multi-array probe incorporates a button 910 in the probe housing to provide control over imaging functionality and grip features 908 for holding the probe. The probe housing 900 incorporates a printed circuit board 911 which provides a microprocessor that interfaces to button 910, provides a motion sensor integrated circuit, and enables digital serial communication through the probe cable 101 to the host processor of the ultrasound imaging system.

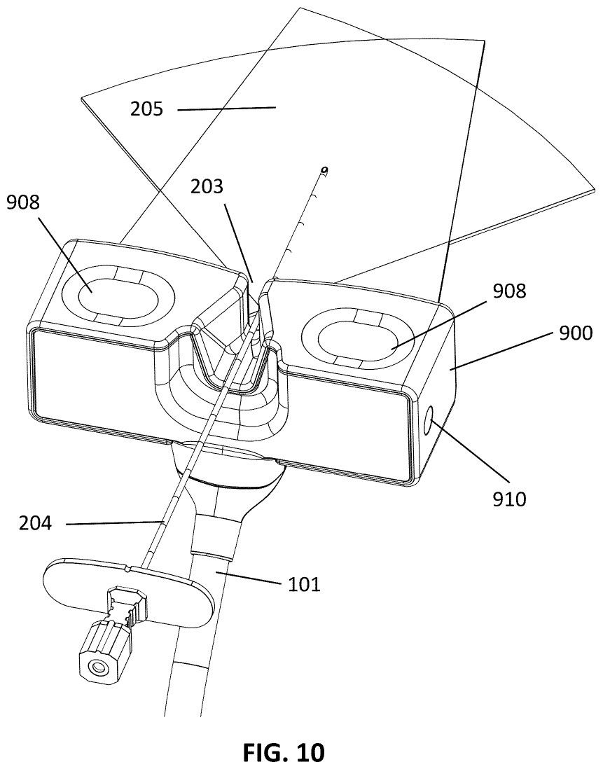

FIG. 10 depicts a representation of the multi-array ultrasound probe from FIGS. 9A-9B as seen by the user during an interventional procedure. In this embodiment, the needle trajectory 204 passes through the medical instrument guide 203, and the configuration of the medical instrument guide 203, probe housing 900, grip features 908, button 910, and ultrasound cable 101 provides an unobstructed view of the needle as it passes through the medical instrument guide 203 and into the patient anatomy and clearance on the rear of the ultrasound probe for manipulating the needle. In this imaging configuration, the fields of view (FOV) 205 of the two ultrasound arrays overlap along the needle trajectory 204, providing for independent views of the needle from two different vantage points.

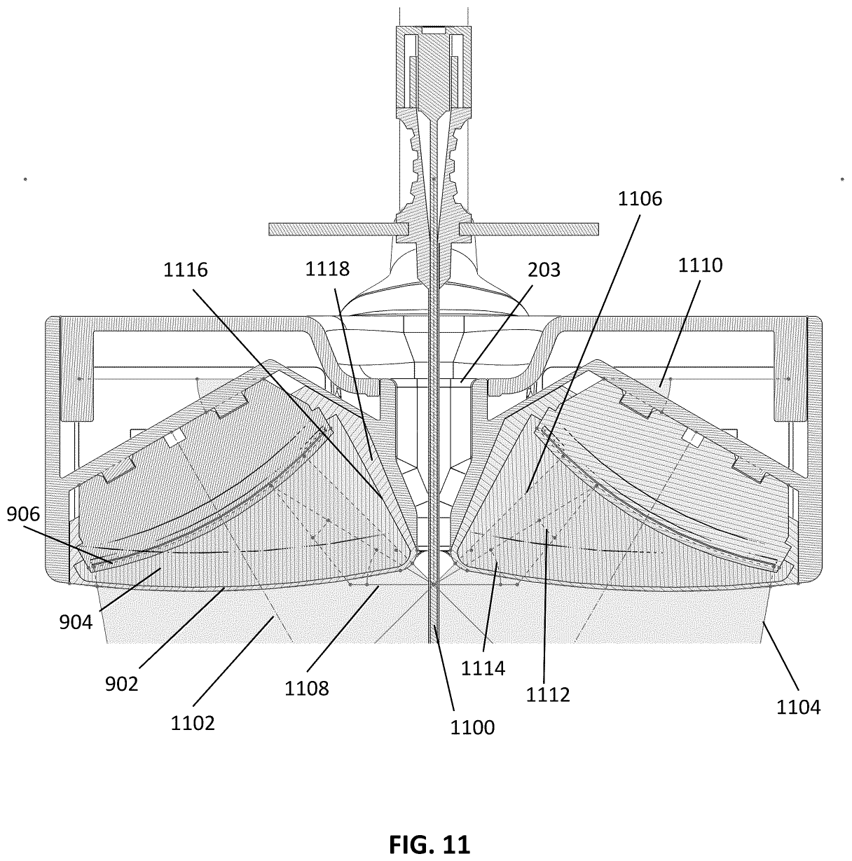

FIG. 11 illustrates an exemplary embodiment that improves needle visibility compared to the embodiment of FIG. 4 by decreasing the acoustic angle of incidence relative to the medical instrument. The multi-array ultrasound probe of FIG. 9 is depicted along with acoustic imaging planes captured by each of two ultrasound transducer arrays 202 and a needle 1100 is present in the medical instrument guide 203. Each of the two ultrasound transducer arrays 202 transmit sound along ultrasound transducer array central axis 1102 and has an outer acquisition limit 1104 and an inner acquisition limit 1106, between which the acoustic energy transmits into the subject anatomy being imaged. The acoustic intensity of the needle in the ultrasound images is dependent on the acoustic angle of incidence relative to the needle 1100. The acoustic angle of incidence relative to the needle 1100 varies with depth, but a primary measurement point of relevance exists at the most superficial acoustic needle depth 1108 contained within the range of the outer and inner acquisition limits 1104, 1106. At the most superficial acoustic needle depth 1108, the minimum acoustic angle of incidence 1114 serves as a determining factor in needle visualization capabilities, with a large minimum acoustic angle of incidence 1114 (such as, by way of example only, greater than 75 degrees) producing limited needle visualization capabilities and a small minimum acoustic angle of incidence 1114 (such as, by way of example only, less than 50 degrees) producing surprisingly improved visualization, with 0 degrees (a needle orthogonal to the axis of sound propagation 1102) producing, in aspects, strong reflections and visibility (in some cases, the strongest reflections and best visibility). In some embodiments, one or more of the ultrasound transducer arrays 202 are rotated by an array rotation angle 1110 to produce a lower minimum acoustic angle of incidence 1114 relative to arrays placed parallel with the subject's skinline (as shown in, e.g., FIG. 4). In some embodiments, one or more of the ultrasound transducer arrays use multi-angle beam steering to transmit and receive ultrasound energy along an angle 1112 relative to the ultrasound transducer array central axis 1102, reducing the minimum acoustic angle of incidence 1114. When the one or more ultrasound transducer arrays 202 are rotated and configured with one or more acoustically transmissive layers to ‘fill’ the space remaining between the ultrasound transducer arrays 202 and the patient anatomy, such as the central acoustically transmissive layer 904, the outermost transmissive layer 902 and the innermost transmissive layer 906 depicted in FIG. 11, acoustic reverberation caused by reflections inside the probe housing can degrade the resulting ultrasound images and obscure or distort anatomical information. In some embodiments, the surface of the acoustically transmissive layer that abuts the instrument guide 1116 is angled non-parallel to the elevation direction of the one or more ultrasound transducer arrays 202 so that acoustic reflections are steered away from the one or more ultrasound transducer arrays 202. In some embodiments, the material that is situated between the acoustically transmissive layer that abuts the instrument guide 1116 and the medical instrument guide 203 has an acoustic attenuation rate greater than that of the central acoustically transmissive layer 904 and an acoustic impedance within 50% of the central acoustically transmissive layer 904.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

As used herein, the term “medical instrument retention components” refers to clips, bands, straps, pins, and buckles, as well as any component that constrains motion of the medical instrument, and as would be understood by one of ordinary skill in the art.

As used herein, the term “medical instruments” refers to needles, catheters, trocars, ablation instruments, cutting instruments, therapy applicators, and other medical instruments as would be understood by one of ordinary skill in the art.

1) An ultrasound imaging apparatus comprising:
a probe housing comprising two or more ultrasound transducer arrays;
wherein the two or more ultrasound transducer arrays are separated by a physical gap of at least 1 mm;
wherein the physical gap is positioned to provide for insertion of a medical instrument with an in-plane orientation relative to an ultrasound imaging plane, and wherein the physical gap is dimensioned for accepting a medical instrument for insertion into a patient anatomy;
wherein the probe housing further comprises a medical instrument guide in or near the physical gap.

2) The ultrasound imaging apparatus of claim 1, wherein the two or more ultrasound transducer arrays are separated from the patient anatomy by one or more intervening acoustically transmissive layers.

3) The ultrasound imaging apparatus of claim 2, wherein the two or more ultrasound transducer arrays do not make direct physical contact with the patient anatomy.

4) The ultrasound imaging apparatus of claim 2, wherein the one or more intervening acoustically transmissive layers comprise a patient contact interface and an acoustic filler material.

5) The ultrasound imaging apparatus of claim 4, wherein the patient contact interface is comprised of one or more rigid, semi-rigid, or substantially rigid materials.

6) The ultrasound imaging apparatus of claim 4, wherein the patient contact interface is comprised of one or more elastic, semi-elastic, or substantially elastic materials.

7) The ultrasound imaging apparatus of claim 4, wherein the one or more intervening acoustically transmissive layers comprises an ultrasound transducer array lens coating.

8) The ultrasound imaging apparatus of claim 2, wherein one or more surfaces of the intervening acoustically transmissive layers is angled non-parallel to an elevational plane of the one or more ultrasound transducer arrays.

9) The ultrasound imaging apparatus of claim 2, wherein a material in contact with one or more surfaces of the one or more intervening acoustically transmissive layers has an acoustic attenuation rate greater than at least one of the one or more intervening acoustically transmissive layers.

10) The ultrasound imaging apparatus of claim 2, wherein a material in contact with one or more surfaces of the one or more intervening acoustically transmissive layers has an acoustic impedance within 50% of at least one of the one or more intervening acoustically transmissive layers.

11) The ultrasound imaging apparatus of claim 1, wherein each of the two or more ultrasound transducer arrays are configured to provide an acoustic angle of incidence of at most 75 degrees at a shallowest point of intersection between the medical instrument and the ultrasound imaging plane.

12) The ultrasound imaging apparatus of claim 1, wherein each of the two or more ultrasound transducer arrays are configured to provide an acoustic angle of incidence of at most 50 degrees at a shallowest point of intersection between the medical instrument and the ultrasound imaging plane.

13) The ultrasound imaging apparatus of claim 1, wherein the medical instrument guide is configured within the probe housing to enable a midline medical instrument insertion trajectory.

14) The ultrasound imaging apparatus of claim 1, wherein the medical instrument guide is integral to the probe housing.

15) The ultrasound imaging apparatus of claim 1, wherein the medical instrument guide allows a medical instrument insertion trajectory angulation of 20 degrees or less relative to the central axis of the medical instrument guide.

16) The ultrasound imaging apparatus of claim 1, wherein the medical instrument guide detachably interfaces with the probe housing to enable a paramedian and/or oblique medical instrument insertion trajectory.

17) The ultrasound imaging apparatus of claim 1, wherein a central axis of sound propagation of each of the two or more ultrasound transducer arrays differs.

18) The ultrasound imaging apparatus of claim 1, wherein a direction of sound propagation of each of the two or more ultrasound transducer arrays creates an overlapping view plane over a target location within the patient anatomy.

19) The ultrasound imaging apparatus of claim 18, wherein the overlapping view plane includes a point where the medical instrument enters the patient anatomy.

20) The ultrasound imaging apparatus of claim 18, wherein a processor is configured to geometrically reconstruct images from the two or more ultrasound transducer arrays by compounding data at spatial locations sampled by more than one ultrasound transducer array of the two or more ultrasound transducer arrays.

21) The ultrasound imaging apparatus of claim 1, wherein a processor controls image acquisition by the two or more ultrasound transducer arrays, and wherein the processor interleaves image acquisition such that the two or more ultrasound transducer arrays are not acquiring images at a same time.

22) The ultrasound imaging apparatus of claim 21, wherein an ultrasound transducer array of the two or more ultrasound transducer arrays acquiring images is the same ultrasound transducer array that transmits ultrasound energy into the patient anatomy.

23) The ultrasound imaging apparatus of claim 21, wherein the ultrasound transducer array of the two or more ultrasound transducer arrays acquiring images is different from the ultrasound transducer array of the two or more ultrasound transducer arrays that transmits ultrasound energy into the patient anatomy.

24) The ultrasound imaging apparatus of claim 1, wherein a processor controls a propagation direction of sound waves from the two or more ultrasound transducer arrays at one or more non-zero angles relative to a central axis of the probe housing, the two or more ultrasound transducer arrays, or both, using electronic beam steering.

25) The ultrasound imaging apparatus of claim 1, wherein a medical instrument retention component mechanically interfaces with the probe housing and the medical instrument guide to restrict the medical instrument to in-plane trajectories.

26) The ultrasound imaging apparatus of claim 1, wherein a medical instrument retention component mechanically interfaces with the probe housing and the medical instrument guide to preserve alignment of the medical instrument with a target location within the patient anatomy.

27) The ultrasound imaging apparatus of claim 1, wherein a medical instrument retention component detachably interfaces with the probe housing and provides a quick-release mechanism for separating the medical instrument from the probe housing.

28) The ultrasound imaging apparatus of claim 1, further comprising a processor, wherein the processor implements a medical instrument detection algorithm to detect image samples depicting the medical instrument.

29) The ultrasound imaging apparatus of claim 28, wherein the processor conveys and overlays a medical instrument location on a graphical display unit via pixel intensity adjustment, pixel coloration, graphical overlays, or combinations thereof, upon an ultrasound image displayed on the graphical display unit.

30) The ultrasound imaging apparatus of claim 28, wherein the medical instrument is a hyperechoic medical instrument, and wherein the medical instrument detection algorithm differentiates between the hyperechoic medical instrument and tissue echoes on a basis of spatiotemporal analysis of sequential image data to detect samples that correspond regions of intensity and motion trajectories consistent with insertion of the medical instrument in the patient anatomy.

31) The ultrasound imaging apparatus of claim 28, wherein the processor adaptively reconfigures a direction of sound propagation of the two or more ultrasound transducer arrays to optimize sensitivity to the medical instrument based on output of the medical instrument detection algorithm.

32) The ultrasound imaging apparatus of claim 1, wherein the probe housing comprises electronic components that measure changes in a spatial position of the two or more ultrasound transducer arrays along the patient anatomy between image acquisitions.

33) The ultrasound imaging apparatus of claim 32, wherein a processor reconstructs volumetric image data from a series of two-dimensional images with discrete spatial positions as captured by the two or more ultrasound transducer arrays.

34) The ultrasound imaging apparatus of claim 32, wherein the volumetric image data are rendered to a graphical display unit.

35) The ultrasound imaging apparatus of claim 1, wherein the probe housing comprises one or more user grip element located to avoid obstructing a medical instrument insertion trajectory.

36) The ultrasound imaging apparatus of claim 35, wherein the one or more user grip element is located to avoid obstructing a visual path to a point at which the medical instrument contacts the patient anatomy.

37) The ultrasound imaging apparatus of claim 35, wherein the medical instrument is one or more of a needle, a catheter, a trocar, an ablation instrument, a cutting instrument, or a therapy applicator.

38) The ultrasound imaging apparatus of claim 1, wherein the probe housing comprises one or more button element located to avoid obstructing a medical instrument insertion trajectory.

39) The ultrasound imaging apparatus of claim 38, wherein the one or more button element is located to avoid obstructing a visual path to a point at which the medical instrument contacts the patient anatomy.

40) The ultrasound imaging apparatus of claim 38, wherein the medical instrument is one or more of a needle, a catheter, a trocar, an ablation instrument, a cutting instrument, or a therapy applicator.
